(19) United States
(12) Patent Application Publication
(10) Pub. No.: US 2009/0088444 A1
(43) Pub. Date: Apr. 2, 2009

(54) Substituted phenanthrene diketo acids and uses therefor

(76) Inventor: John K. Buolamwini, Cordova, TN (US)

Correspondence Address: Dr. Benjamin Adler;ADLER & ASSOCIATES, 8011 Candle Lane, Houston, TX, 77071, US

(21) Appl. No.: 12/286,820

(22) Filed: Oct. 2, 2008

Related U.S. Application Data
(60) Provisional application No. 60/997,212, filed on Oct. 2, 2007.

(51) Int. Cl. A61K 31/495; C07D 295/112; C12N 9/99; C12N 9/00; C12Q 1/68

(52) U.S. Cl. 514/255.03; 544/381; 435/184; 435/4; 435/6

(57) ABSTRACT

Provided herein are substituted phenanthrene diketo acid compounds. These compounds comprise the diketo acid moiety on one of carbons C1-C4 and C9 in the phenanthrene ring and at least one further substitutent on the other ring carbons. Also provided are methods of inhibiting an activity of a human immunodeficiency virus (HIV) integrase protein and treating an HIV infection in a subject using the substituted phenanthrene diketo acid compounds.

### CROSS-REFERENCE TO RELATED APPLICATION

This non-provisional application claims benefit of provisional application U.S. Ser. No. 60/997,212 filed on Oct. 2, 2007, now abandoned.

### BACKGROUND OF THE INVENTION

1. Field of the Invention

The present invention relates to the fields of organic chemistry, enzymology and molecular biology. Specifically, the present invention relates to integrase inhibitors and uses therefor in treating human immunodeficiency viral (HIV) infections.

2. Description of the Related Art

Acquired immunodeficiency syndrome (AIDS), a disease resulting from infection with human immunodeficiency virus (HIV), is one of the world's most serious health problems. It is estimated that approximately 39 million people are living with HIV/AIDS worldwide, with an infection and death rates of around 4 million and 3 million per year, respectively (1). Three essential enzymes are encoded by the HIV pol gene, i.e., reverse transcriptase (RT), integrase (IN) and protease (PR)2, have been the subjects for anti-HIV drug development among others. Drugs targeting RT and PR have been available for over a decade and have shown efficacy particularly when employed in combination (3-5). However, infection still cannot be eradicated completely with current highly active antiretroviral therapy (HAART) combination treatments and toxicity and drug resistance are problems as well (6-7).

Thus, there is a need for new inhibitors that could block the virus at other steps of its replication cycle. HIV integrase (IN) is an attractive potential drug target in this regard because it is responsible for incorporation of HIV provirus into the host cell genome, is essential for viral replication, and does not have a direct human counterpar (8-11). The pertinent catalytic activity of IN involves the cleavage of a dinucleotide fragment from each end of the proviral DNA, i.e., 30-end processing, and insertion of this donor DNA into the host cellular DNA, i.e., strand transfer (12-14). For almost the past decade and half, many integrase inhibitors with diverse structural features have been identified, designed, synthesized, and screened for the inhibitory activity, and the results show that effective inhibition is possible (15-20).

There is still, however, a recognized need in the art for improved HIV therapeutics and therapies. Specifically, the prior art is deficient in substituted phenanthrene diketo acids effective to inhibit HIV-1 integrase activity and viral replication. The present invention fulfills this long standing need in the art.

### SUMMARY OF THE INVENTION

The present invention is directed to a phenanthrene diketo acid compound having the structure

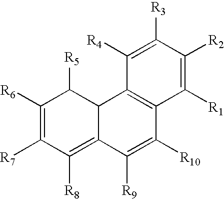

The R₁ substituents may be —C(O)CH₂C(O)COOH or H, the R₂ substituents may be H, F, —OCH₃, or NH₂, the R₃ substituents may be R₁ or F, the R₄ substituents may be R₂, R₃, OH, CH₃, or NHC(O)CH₃, the R₅ substituents may be H, F or C₁-C₄alkoxy, the R₆ substituents may be H, F, CN, C₁-C₄alkyl, C₁-C₄alkoxy, or O(CH₂)₃CN, the R₇ substituents may be H, F, CF₃, NO₂, C₁-C₄alkoxy, piperidyl, or methylpiperazine, the R₈ substituents may be H, NO₂ or C₁-C₄alkoxy, the R₉ substituents may be R₁, and the R₁₀ substituents may be CH₂OCH₂OCH₃, H, C₁-C₅alkyl, C₁-C₄alkoxy, tetrahydrofuran, 1-methyl-2,4-cyclopentene, pyrrolidine, or pyridine, where one of R₁, R₂, R₃, R₄ or R₉ is C(O)CH₂C(O)COOH.

The present invention also is directed to a synthetic inhibitor of HIV integrase protein comprising a phenanthrene ring substituted with a diketo acid moiety at one of C1-C4 and C9 on the phenanthrene, said phenanthrene ring further substituted with other than a hydrogen at one or more of carbons C1-C10.

The present invention is directed further to a method for inhibiting an activity of a human immunodeficiency viral (HIV) integrase protein. The method comprises contacting an HIV virus or a cell comprising HIV with the PKA compounds described herein thereby inhibiting integrase protein activity therein.

The present invention is directed further still to a method for treating an HIV infection in a subject. The method comprises administering to the individual a pharmacologically effective amount of one or more PKA compounds described herein to the subject thereby treating the HIV infection.

The present invention is directed further still to a method for identifying an inhibitor of HIV integrase protein. The method comprises designing a test compound based on the PKA compounds as lead compounds and measuring a level of an HIV integrase protein activity in the presence and the absence of the test compound. The HIV integrase protein activity level in the presence of the test compound is compared with the HIV integrase activity level in the absence of the test compound where a decrease in HIV integrase protein activity in the presence of the test compound is indicative that the test compound is an inhibitor of HIV integrase protein. The present invention is directed to a related method further comprising determining a therapeutic index for the inhibitor. Also, the present invention is directed to a related inhibitory compound identified by the method described herein.

Other and further aspects, features and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIGS. 1A-1H depict the synthetic schema 1-8 used to synthesize the diketo acid compounds disclosed herein.

FIGS. 2A-2C depict the substituents for the aryl-methyl ketone starting materials used to synthesize substituted phenanthrene diketo acids.

### DETAILED DESCRIPTION OF THE INVENTION

As used herein, the term “a” or “an”, when used in conjunction with the term “comprising” in the claims and/or the specification, may refer to “one”, but it is also consistent with the meaning of “one or more”, “at least one”, and “one or more than one”. Some embodiments of the invention may consist of or consist essentially of one or more elements, method steps, and/or methods of the invention. It is contemplated that any device, compound, composition, or method described herein can be implemented with respect to any other device, compound, composition, or method described herein.

As used herein, the term “or” in the claims refers to “and/or” unless explicitly indicated to refer to alternatives only or the alternatives are mutually exclusive, although the disclosure supports a definition that refers to only alternatives and “and/or”.

As used herein, the term “compound” is interchangeable with “inhibitor”, or “inhibitory compound” and means a molecular entity of natural, semi-synthetic or synthetic origin that blocks, stops, inhibits, and/or suppresses substrate interactions with HIV integrase protein or peptide.

As used herein, the term “contacting” refers to any suitable method of bringing one or more of the compounds described herein or other inhibitory agent into contact with an HIV integrase protein or peptide, as described, or a cell comprising the same. In vitro or ex vivo this is achieved by exposing the HIV integrase protein or peptide or cells comprising the same to the compound or inhibitory agent in a suitable medium. For in vivo applications, any known method of administration is suitable as described herein.

As used herein, the terms “effective amount” or “pharmacologically effective amount” are interchangeable and refer to an amount that results in an improvement or remediation of the symptoms of a disease, disorder or condition. Those of skill in the art understand that the effective amount may improve the patient's or subject's condition, but may not be a complete cure of the disease, disorder and/or condition.

As used herein, the term “inhibit” refers to the ability of the compound to block, partially block, interfere, decrease, reduce or deactivate HIV integrase. Thus, one of skill in the art understands that the term inhibit encompasses a complete and/or partial loss of activity of integrase. Integrase activity may be inhibited by occlusion or closure of the docking domain, by disruption of the interaction with the substrate, by sequestering integrase and/or the substrate, or by other means. For example, a complete and/or partial loss of activity of integrase may be indicated by a reduction in 3′ processing and strand transfer of viral DNA, a reduction in viral replication, or the like.

As used herein, the term “treating” or the phrase “treating a human immunodeficiency viral infection (HIV)” or “treating an HIV infection” includes, but is not limited to, halting the replication of HIV or reducing the viral load. Treating HIV encompasses therapeutic administration of the inhibitor(s) described herein singly or in combination with other known HIV therapeutic agents or pharmaceuticals.

As used herein, the term “subject” refers to any recipient of an integrase inhibitor, optionally including other known HIV therapeutic agents or pharmaceuticals as a treatment for HIV infection or a treatment given for a similar purpose as described herein.

In one embodiment of the present invention, there is provided a phenanthrene diketo acid (PKA) compound having the structure

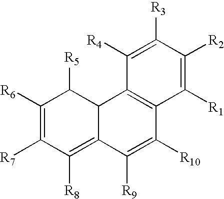

wherein R₁ is —C(O)CH₂C(O)COOH or H; R₂ is H, F, —OCH₃, or NH₂; R₃ is R₁ or F; R₄ is R₂, R₃, OH, CH₃, or NHC(O)CH₃; R₅ is H, F or C1-C₄alkoxy; R₆ is H, F, CN, C₁-C₄alkyl, C₁-C₄alkoxy, or O(CH₂)₃CN; R₇ is H, F, CF₃, NO₂, C₁-C₄alkoxy, piperidyl, or methylpiperazine; R₈ is H, NO₂ or C₁-C₄alkoxy; R₉ is R₁; and R₁₀ is CH₂OCH₂OCH₃, H, C₁-C₅alkyl, C₁-C₄alkoxy, tetrahydrofuran, 1-methyl-2,4-cyclopentene, pyrrolidine, or pyridine; where one of R₁, R₂, R₃, R₄ or R₉ is C(O)CH₂C(O)COOH.

In this embodiment the phenanthrene diketo acid compound may be a pharmacologically effective salt or hydrate thereof. Further to these embodiments the PKA compound may be a pharmaceutical composition further comprising a pharmaceutically effective carrier.

In one aspect of these embodiments R₁ may be C(O)CH₂C(O)COOH, R₂ may be H or NH₂, R₃ may be H, R₄ is F, CH₃, OCH₃, R₅ may be H or OCH₃, R6 may be H, CN, OCH₃, OCH₂CH₃, or O(CH₂)₃CN, R₇ may be H, NO₂, CF3, CH₃, OCH₃, OCH₂CH₃, piperidyl, or methylpiperazine, R₈ may be H or OCH₃, and R₉ and R₁₀ may be H.

In another aspect of these embodiments R₁, R₅-R₆ and R₈-R₁₀ may be H, R₂ may be C(O)CH₂C(O)COOH, R₃ and R₄ may be H or F, and R₇ may be CH₃.

In yet another aspect of these embodiments R₁-R₂ and R₈-R₁₀ may be H, R₃ may be C(O)CH₂C(O)COOH, R4 may be H or F, R5 may be H, F, OCH₃, or O(CH2)3CH3, R₆ may be H, F, CN, CH₃, CH(CH₃)₃, OCH₃, OCH₂(CH₃)₂, O(CH₂)₃CH₃, O(CH₂)₃CN, and R₇ may be H, F, CF₃, NO₂, CH₃, OCH₃, piperidyl, or methylpiperazine.

In yet another aspect of these embodiments R₁-R₃, R₅-R₆ and R₈-R₁₀ may be H, R₄ may be C(O)CH₂C(O)COOH and R₇ may be CH₃.

In yet another aspect of these embodiments R₁-R₈ may be H, R₉ may be C(O)CH₂C(O)COOH and R10 may be CH₂OCH₂OCH₃, H, C₁-C₅alkyl, C₁-C₄alkoxy, tetrahydrofuran, 1-methyl-2,4-cyclopentene, pyrrolidine, or pyridine.

In another embodiment of the present invention there is provided a synthetic inhibitor of HIV integrase protein comprising a phenanthrene ring substituted with a diketo acid moiety at one of C1-C4 and C9 on the phenanthrene, said phenanthrene ring further substituted with other than a hydrogen at one or more of carbons C1-C10. In this embodiment one or more of carbons C1-10 may be substituted with one or more of fluorine, trifluoromethyl, nitrite, amine or alkylamine, alkyl or alkoxy or cyano-alkoxy, an alkyl diether, piperidyl, methylpiperazinyl, tetrahydrofuranyl, methylcyclopentenyl, pyrrolidinyl, or pyridinyl.

In yet another embodiment of the present invention there is provided a method for inhibiting an activity of a human immunodeficiency viral (HIV) integrase protein, comprising contacting an HIV virus or a cell comprising HIV with the compound described supra thereby inhibiting integrase protein activity therein. In this embodiment the HIV integrase activity may be 3′-end processing or strand transfer of HIV RNA. In this embodiment the human immunodeficiency virus may be HIV type 1 (HIV-1).

In yet another embodiment of the present invention there is provided a method for treating an HIV infection in a subject, comprising administering to the individual a pharmacologically effective amount of one or more compounds described supra to the subject thereby treating the HIV infection. Further to this embodiment the method comprises administering one or more other HIV antiviral drugs to the individual. In this further embodiment the other HIV antiviral drug(s) is administered concurrently with or sequentially to the administration of the compound(s). In both embodiments the human immunodeficiency virus may be HIV type 1 (HIV-1).

In yet another embodiment of the present invention there is provided a method for identifying an inhibitor of HIV integrase protein, comprising designing a test compound based on the compounds described supra as lead compounds; measuring a level of an HIV integrase protein activity in the presence and the absence of the test compound; and comparing the HIV integrase protein activity level in the presence of the test compound with the HIV integrase activity level in the absence of the test compound, wherein a decrease in HIV integrase protein activity in the presence of the test compound is indicative that the test compound is an inhibitor of HIV integrase protein. Further to this embodiment the method comprises determining a therapeutic index for the inhibitor. The HIV integrase activity type of HIV is as described supra.

Provided herein are synthetic substituted phenanthrene diketo acid compounds, including derivatives and analogs thereof. These phenathrene diketo acids exhibit inhibitory effects against human immunodeficiency virus. Without being limiting, for example, the compounds provided herein are effective against an HIV integrase activity and/or viral replication. For example, the synthetic compounds are effective to inhibit 3′-end processing and/or strand transfer of HIV RNA into a host DNA in vitro or in vivo.

Generally, the inhibitory compounds comprise a phenanthrene skeleton having a diketo acid moiety at one of positions C1-C4 and C9 on the aromatic ring. The one or more of the carbons not comprising the diketo acid moiety may comprise substituents such as, inter alia, hydrogen, fluorine, trifluoromethyl, nitro, amine or short chain alkylamine, straight- or branched-chain alkyl or alkoxy or cyano-alkoxy, an alkyl diether, or heterocycles such as piperidyl, methylpiperazinyl, tetrahydrofuranyl, methylcyclopentenyl, pyrrolidinyl, or pyridinyl. These compounds are synthesized from the corresponding methyl ketone where one of C1-C4 or C9 is substituted with an acetyl moiety as described in the Examples herein. Preferred aryl methyl ketones are identified in Tables 1-3.

It is contemplated that the inhibitor compounds described herein may be useful as lead compounds in the design of derivative and analog compounds, including computer-aided design. Alternatively, screening chemical libraries may be screened for structurally similar substituted phenanthrene compounds or analogs, as is known in the art. Potential compounds may be synthesized using the methods described herein or other chemical synthetic methods suitable for the proposed structures. Efficacy of these designed test compounds may be determined using the assays described herein or other assays suitable to determine activity of HIV integrase protein or a peptide thereof or the replication efficiency of the virus. In addition the therapeutic index of the identified inhibitors may be determined by standard methods known to those skilled in the art.

Thus, the integrase inhibitors provided herein are useful as therapeutics. The inhibitory compounds provided herein may be used to treat any subject, preferably a human, having an HIV infection, such as, but not limited to subtype HIV-1. It is contemplated that contacting the HIV virus with one or more of these compounds is effective to at least inhibit, reduce or prevent HIV integrase activity and/or HIV replication in cells. The compounds of the present invention may be administered alone or in combination or in concurrent therapy with other therapeutic agents or pharmaceuticals which affect HIV.

The present invention also contemplates therapeutic methods employing compositions comprising the active substances disclosed herein. Preferably, these compositions include pharmaceutical compositions comprising a therapeutically effective amount of one or more of the active compounds or substances along with a pharmaceutically acceptable carrier. Also, these compositions include pharmacologically effective salts or hydrates of the inhibitors.

As is well known in the art, a specific dose level of active compounds, such as integrase inhibitors or related derivative or analog compounds thereof for any particular patient depends upon a variety of factors including the activity of the specific compound employed, the age, body weight, general health, sex, diet, time of administration, route of administration, rate of excretion, drug combination, and the severity of the HIV infection undergoing therapy. The person responsible for administration is well able to determine the appropriate dose for the individual subject and whether a suitable dosage of either or both of the inhibitory compound(s) and other HIV therapeutic agent(s) comprises a single administered dose or multiple administered doses.

The following example(s) are given for the purpose of illustrating various embodiments of the invention and are not meant to limit the present invention in any fashion.

### EXAMPLE 1

### General Methods

### Chemistry

Two multi-vessel reactions carousels (Radeleys Discovery Technologies), available for parallel solution synthesis, are fitted with Teflon stoppers and have temperature control and magnetic stirrer blocks, as well as inlet and outlet facilities for introduction of inert gases to maintain a proper reaction atmosphere. Because traditional heating techniques are slow and time-consuming and can lead to decomposition of the substrate and/or product and low reaction yields, a microwave multi-station parallel synthesizer is used to reduce the reaction times from hours to minutes and to increase yields and selectivity (21). A single reaction station microwave-assisted organic synthesizer (CEM Corporation) is used for developing reactions before applying to the multi-station microwave parallel synthesizer.

### Separation, Purification and Structure Elucidation

Silica gel thin layer chromatography is used to follow reaction progress. Flash silica gel column chromatography is used for general separation and purification where extractions and recrystalization are not effective. Prepacked multi-column systems are used for simultaneous purification of reactions from parallel synthesis. Reverse-phase HPLC is used where necessary. Melting points are determined, IR spectra recorded on FT-IR instrument; high resolution ¹H and ¹³C NMR spectra are recorded on Varian 300 or 500 MHz instruments; mass spectra are recorded on an electrospray instrument coupled to a liquid chromatography system. Purity is analyzed by elemental analysis for carbon, nitrogen and hydrogen (Atlanta Microlabs, Norcross, Ga.). The standard purity levels of elemental composition deviations of not more that 0.4% are used as cut-off for purity. HPLC is used to determine the purity for products obtained in very small quantities and a purity level above 95% is maintained.

### Testing Compounds as Inhibitors in Integrase Assays

Briefly, a 21 amino acid top sequence 5′-GTGTGGAAAATCTCTAGCAGT-3′ (SEQ ID NO: 1) and a 21 amino acid bottom sequence 5′-ACTGCTAGAGATTTTCCACAC-3′ (SEQ ID NO: 2) (Norris Cancer Center Core Facility, University of Southern California) are purified by UV shadowing on polyacrylamide gel. To analyze the extent of 3′-processing and strand transfer using 5′-end labeled substrates, SEQ ID NO: 1 is 5′-end labeled using T₄ polynucleotide kinase (Epicenter, Madison, Wis.) and gamma-[³²P]-ATP (Amersham Biosciences). The SEQ ID NO:2 oligo is added to the heat-inactivated kinase which is in 1.5-molar excess. The mixture is heated at 95° C., is allowed to cool slowly to room temperature and is run through a spin 25 mini-column (USA Scientific) to separate annealed double-stranded oligonucleotide from unincorporated material.

To determine the extent of 3′-processing and strand transfer, HIV-1 integrase is preincubated at a final concentration of 200 nM with the inhibitor in reaction buffer (50 mM NaCl, 1 mM HEPES, pH 7.5, 50 mM EDTA, 50 mM dithiothreitol, 10% glycerol (w/v), 7.5 mM MnCl₂, 0.1 mg/mL bovine serum albumin, 10 mM 2-mercaptoethanol, 10% DMSO, and 25 mM MOPS, pH 7.2) at 30° C. for 30 min. Then, 20 nM of the 5′-end ³²P-labeled linear oligonucleotide substrate is added, and the incubation continued for an additional 1 h. Reactions are quenched by addition of 8 mL of loading dye (98% deionized formamide, 10 mM EDTA, 0.025% xylene cyanol, and 0.025% bromophenol blue). An aliquot (5 mL) is electrophoresed on a denaturing 20% polyacrylamide gel (0.09 M tris-borate pH 8.3, 2 mM EDTA, 20% acrylamide, 8 M urea). Gels are dried, exposed on a Phosphorlmager cassette, read on a Typhoon 8610 Variable Mode imager (Amersham Biosciences), and quantitations are performed using ImageQuant 5.2 (Amersham Biosciences).

### Testing Compounds as Inhibitors of HIV Replication in Cell Culture and Cytotoxicity Testing

Anti-HIV-1 activity in cell culture is conducted using peripheral blood mononuclear cells (PBMCs). PBMCs at 10⁷ cells/T25flask are stimulated with phytohemagglutinin for 3 days and are infected with a wild-type HIV-1 strain (strain LAI) at 100 50% tissue culture infective doses, as previously described (22). The cultures were kept for 5 days in the presence of test compounds at serial 1-log dilutions. Subsequently, human PBMCs are removed from the culture supernatant by 10 min centrifugation at 400×g, at 4° C. The clarified supernatant is analyzed by a reverse transcriptase assay.

To determine therapeutic index the compounds are tested for cytotoxicity using uninfected PBMCs, CEM leukemia and Vero African green monkey kidney cells, according to a previous method (23). PBMCs are obtained from whole blood of healthy individuals, while CEM and Vero cells (American Type Tissue Collection, Rockville, Md.). The PBMCs and CEM cells are cultured in the presence or absence (controls) of compound for 6 days. After this time period, cells are stained with Trypan blue dye, for cell proliferation and a viability determination is made, as previously described (24). Only the effects on cell growth are reported, since they correlated well with cell viability. For Vero cells the incubation period was 3 days. Promising compounds are tested against drug resistant HIV isolates from HIV/AIDS patients.

### EXAMPLE 2

### Chemical Synthesis

### General Synthesis of New Phenanthrene Diketoacid Derivatives and Analogs

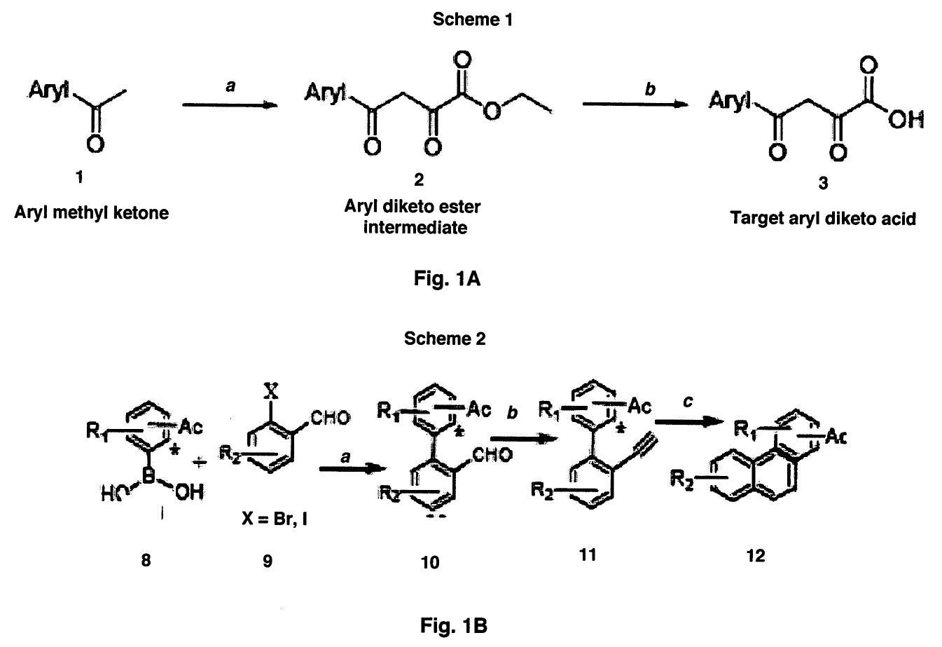

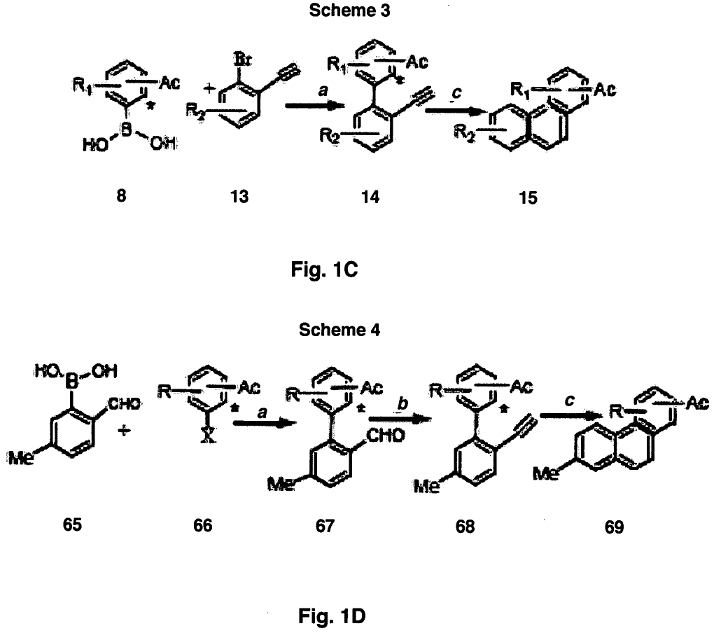

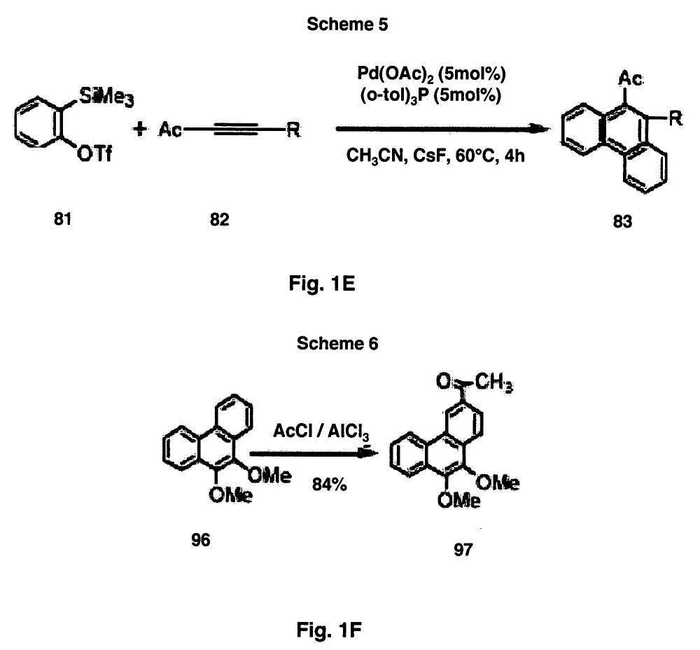

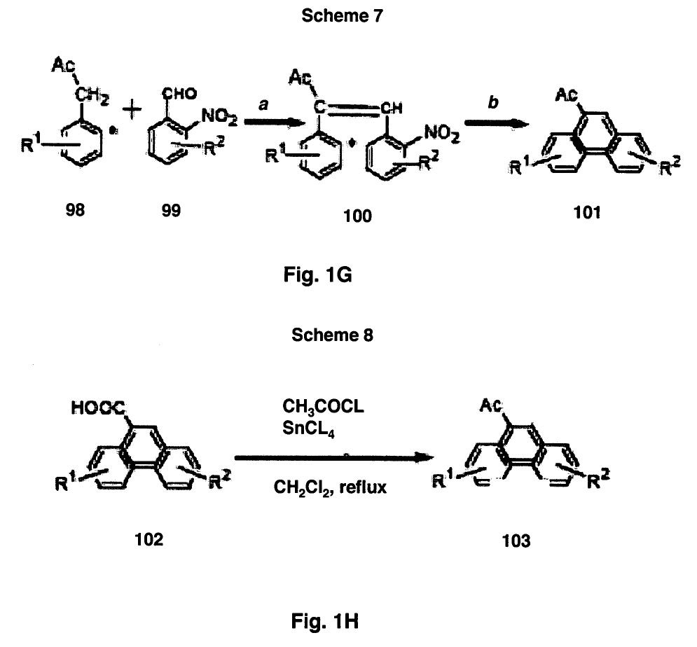

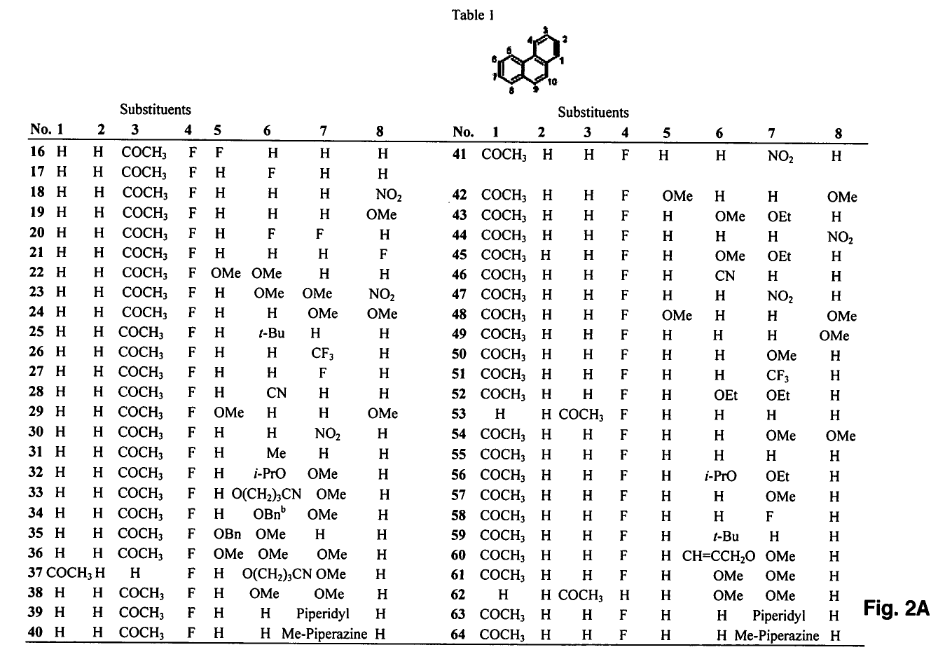

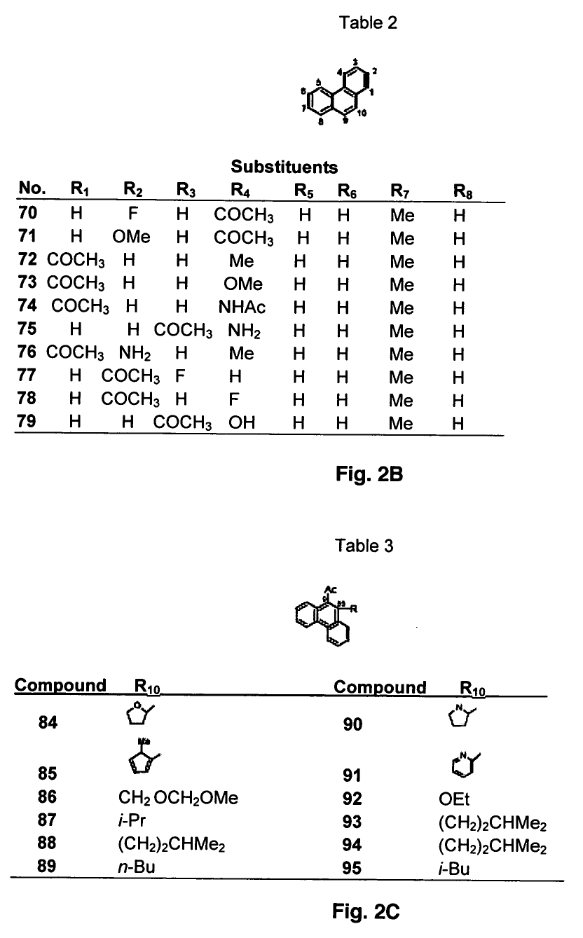

The general synthetic route shown in Scheme 1 (FIG. 1A) is used to synthesize the novel phenanthrene diketo acids. The substituted phenanthrene portions of the proposed compounds are shown in the acetylphenanthrene starting materials listed in Tables 1-3 in FIGS. 2A-2C. Compounds have been designed to provide both conformational/geometric and physicochemical diversity. Synthetic routes for obtaining the starting aryl methyl ketones are also presented.

The appropriate aryl methyl ketone 1 is oxalylated in the presence of base (NaOMe) and diethyl oxalate to give intermediate ethyl esters 2, which are hydrolyzed under alkaline conditions to give the desired free acid target compounds, 3. Good to excellent yields, 55-92%, were obtained. This synthesis depends on the oxalating agent, usually diethyl oxalate or dimethyl oxalate, the base used, usually NaOEt or NaOMe, as well as NaH, temperature and time.

Alternatively, the oxalylating agent tert-butyl methyl oxalate, which has been shown to provide a highly efficient method for synthesizing aryl diketo acids and works well with ketones bearing electron-withdrawing groups, can be used (25). This is particularly suitable for designed phenanthrene diketo acids with electron withdrawing groups, like fluorine, CF₃, and CN, which have been designed to reduce their carcinogenicity. The solvent also may be varied, e.g., THF-DME and THF-MeOH mixtures. In addition acid hydrolysis also may be performed (25).

### EXAMPLE 3

### Synthesis of Substituted Acetylphenanthrene (Phenanthrene Methyl Ketone) Starting Materials

In addition to the commercially available substituted mono acetylphenanthrenes and analog compounds 4-7, diverse substituted acetylphenanthrene starting materials are synthesized, (Schemes 2-7, FIGS. 1B-1G). The starting compounds are oxalylated followed by hydrolysis to afford the desired phenanthrene diketo acids. These phenanthrene diketo acids are tested for HIV integrase inhibition and for inhibition of HIV replication in cell culture. Compounds 4 and 5 allow for functionalization of the C9 and C10 positions by substitution of the bromine atom, if the resulting diketo acids have biological activities. Compound 6 provides for an aza anlog, while compound 7 provides for a partially reduced analog. Benzylic bromination of 7 can be used to functionalize it.

### Synthesis of Diversified Small Molecular Weight Acetylphenanthrene Starting Materials

Several synthetic routes have been designed as shown in the synthetic schemes 2-8 (FIGS. 1B-1H) presented below, for use in the synthesis of the acetylphenanthrene starting materials for the synthesis of target substituted phenantthrene diketo acids and analogs, with some potential compounds shown in Tables 1-3 in FIGS. 2A-2C.

The Suzuki-Miyaura cross-coupling and PtCl₂-catalyzed cycloisomerization reactions are used for synthesis. The facile Suzuki-Miyaura cross-coupling (26) reaction as recently applied by Luliano et al. (27) or Frustner and Mamane (28). Thus the commercially available 2-, 3-, or 4-acetylphenyl, 2-fluoro-3-acetylphenyl or 3-acetyl-6-fluorophenyl boronic acids represented by structure 8 in Scheme 2, are reacted with the commercially available, appropriately substituted 2-bromo or 2-iodobenzaldehyde 9 to obtain the desired acetylbiphenyl aldehyde, 10, according to reactions in synthetic schemes 2-3 (FIGS. 1B-1C).

Compound 10 is then be converted to the alkyne according to reaction b (29) in Scheme 2 by reaction with dimethyl-1-diazo-2-oxopropylphosphonate, which is prepared by treating the commercially available dimethyl-2-oxopropylphosphonate with commercially available TsN₃. This diazo transfer has been shown to take place in excellent yields (>80%) (29). This particular reaction for alkyne formation was chosen rather than the other methods which use strong bases like n-BuLi (30), so as to preserve the necessary acetyl group required on the phenanthrene starting material for synthesizing the desired aryl diketo acids. Another attractive feature of this reaction is the high-yields and simple work up. The terminal aromatic alkyne so formed will then be subjected to PtCl₂-catalyzed cycloisomerization reaction (28, 31), i.e., reaction c in Schemes 2-4 (FIGS. 1B-1D). The second step in the synthetic route can be avoided if a commercially available haloaryl alkyne such as 13 is used, as shown in Scheme 3.

In Scheme 2 (FIG. 1B) R₁ is H, 2-F or 4-F with respect to the Ac (acetyl, COCH₃) group. There are five commercially available suitable acetylphenyl boronic acids of this kind. R₂ represents single or multiple substitutions with F, MeO, alkyl, CN, NO₂, CF₃, etc. FIG. 2A shows substitution patterns for compounds 16-64.

In Scheme 3 (FIG. 1C) R₁ and Ac are as in Scheme 2 (FIG. 1B). R₂ is H or 3,4-di-MeO. In both Schemes 2 and 3 the reagents and conditions are: a)PD(PPh3)4, THF, K2CO3, H2O, reflux; b) dimethyl-1-diazo-20oxopropylphosphonate, K2CO3, MeOH, room temperature; c) PtCl2, catalyst, toluene, 80° C. The asterisk indicates that one ortho-position must be vacant in the staring material to allow for the cyclization at that ring carbon in reaction c.

Synthesis of starting materials with the ortho-formyl boronic acid 65 coupling with the halo acetophenone compounds 66 is performed as according to Scheme 4 (FIG. 1D). This reaction yields various 7-methyl acetylphenanthrene derivatives 69 shown in FIG. 2B.

In Scheme 4, X is Br or I and R is various substituents as shown in FIG. 1B. All three reactions have been reported as high-yielding and highly selective in the literature cited (27-29, 31). However, as the substrates for the Suzuki-Miyaura cross-coupling change, especially when high electron deficient bromo or iodo aldehydes (such as those containing NO₂, CF₃ substituents) in Scheme 2, or halo aryl substrates in Scheme 4, are involved, yields reduce with the original catalysts without good ligand support. In those instances more highly reactive palladium catalyst systems are used. For example, a recently developed highly active catalyst system (32) involving Pd and dialkylbiphenylphosphino ligands such as 80 in which Cy is cyclohexyl.

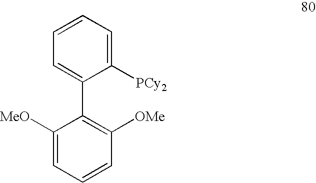

This system allows coupling of pyridine and pyrimidine halides, and works well in the presence of functional groups, electron deficient boronic acids, and even where there is steric hindrance. Although in the cyclization step, reaction c in Schemes 2-4, the overwhelmingly preferred product is the phenanthrene resulting from 6-endo-dig cyclization, when using PtCl₂ as catalyst (5 mol %), there may also be small formation of the 5-exo cyclization product (28), which will be easily separated. Yields up to 90% were obtained. In case PtCl₂ catalysis is not working, AuCl₃ (5 mol %) or GaCl₃ (10 mol %) will be tried catalysts as well (28). If the solvent (toluene) is suitable especially for alkynes with polar substituents, it will be replaced by acetonitrile, which when heated to the same temperature, has also been shown to be suitable for PtCl₂ catalyzed cycloisomerization of alkynes (33). Dichloromethane has also been shown to be a possible suitable solvent, only that it is on the nonpolar side.

### Synthesis of 10-Substituted 9-Acetylphenanthrenes

To further diversify the library of phenanthrene diketo acids, 10-substituted 9-phenanthrene diketoacids are synthesized. The 9-acetylphenanthrene starting materials are synthesized by the facile one step palladium catalyzed co-trimerization of benzyne, deriving from the precursor 81, with appropriately substituted acetyl alkynes 82 as shown in Scheme 5 (FIG. 1E) (34). The commercially available benzyne precursor 2-(trimethylsilyl)phenyl trifluoromethanesulfonate 83 is added to a suspension of anhydrous CsF, Pd(OAc)₂ and (o-tol)₃P in acetonitrile. The commercially available acetyl alkyne is then added and the mixture stirred for 4 h at 60° C. The solvent is evaporated and the residue chromatographed to isolate the phenanthrene product. This catalyst system is specific for obtaining the phenanthrene product in good yields (60-75%) (34). Both electron rich and electron deficient alkynes are tolerated. The acetylphenanthrenes that can be synthesized from commercially materials are shown in FIG. 2C.

### Synthesis of Acetylphenanthrenes by Friedel-Crafts Acetylation

For some acetyl phenanthrene starting materials, the standard Friedel-Crafts acetylation is used to prepare them. Thus, the selected commercially available phenanthrene derivatives are acetylated using acetyl chloride and AlCl₃ in a suitable solvent such as nitrobenzene as shown by example in Scheme 6 (FIG. 1F) for preparing 3-acetyl-9,10-dimethoxyphenanthrene from 9,10-dimethoxyphenanthrene (35). These are one-step reactions which decreases needed synthesis time to prepare aryl methyl ketones.

### Synthesis of 9- or 10-Acetylphenanthrene Starting Materials Using Modified Pschorr Cyclization

To prepare further substituted 9- or 10- acetylphenanthrenes a Pschorr type cyclization is utilized in the reaction sequence shown in Scheme 7 (FIG. 1G).

In Scheme 7 the reagents and conditions are: a) piperdine, benzene, reflux; b) 1. FeSO4, NH4OH, acetone, reflux, 2. I-amylnitrite, HBF4 (485), K4Fe(CN)6, ferrocene (20 mol %), acetone, 0° C. This route allows the synthesis of a wide variety of the 9- or 10-acetylphenanthrene as starting materials. Selected commercially available appropriately substituted phenyl acetones 98 are used and are reacted with the selected commercially available ortho-nitrobenzaldehydes 99 according to literature procedure, reaction a in Scheme 7 (36-37). The resulting a,b-unsaturated ketone 100 then is subjected to a soluble catalyst non-aqueous modification of Pschorr cyclization according to reaction b in Scheme 7 adaptation of the method of Wassmundt and Kiesman (38).

Reaction conditions may have to be modified to improve yields or to enable the reactions. If the use of the substituted phenyl acetones 99 is problematic, the ususl 9- or 10-phenanthrene carboxylic acids are synthesized from phenylacetic acid and ortho-nitrobenzadehyde starting materials to obtain the acid 102 (38-39), which are converted to the desired acetyl starting materials by the following reaction in Scheme 8 (FIG. 1H) according to the method Nordlander and Njoroge (40).

### Converting Commercially Available Phenanthrene Carboxylic Acids to Phenanthrene Methyl Ketone Starting Materials

Scheme 8 (FIG. 1H) is also used to synthesize other acetylphenanthrene starting materials from commercially available appropriately substituted phenanthrene carboxylic acids. This is a very useful reaction, but is limited by the number of commercially available phenanthrene carboxylic acids.

The following references are cited herein.

1. UNAIDS 2006 Report on the Global AIDS Epidemic.

2. Coffin et al. Cold Spring Harbor Laboratory Press: Plainview, N.Y., 1999.

3. Palella et al. (1998) Eng. J. Med. 338:853-860.

4. Bushman et al. (1998) Proc. Natl. Acad. Sci. U.S.A. 95:11041-11042.

5. De Clercq, E. J. (1995) Med. Chem. 38:2491-2517.

6. Zhang et. (1999) N. Eng. J. Med. 340:1605-1613.

7. Furtado et al. (1999) N. Eng. J. Med. 340:1614-1622.

8. Brown, P. O. (1990) Curr. Top. Microbiol. Immunol. 157:19-48.

9. Goff, S. P. (1992) Annu. Rev. Genet. 26:527-544.

10. Katz, R. A. and Skalka, A. M. (1994) Annu. Rev. Biochem. 63:133-173.

11. Farnet et al. (1996) Proc. Natl. Acad. Sci. U.S.A. 93:9742-9747.

12. Bushman, F. D. and Craigie, R. (1991) Proc. Natl. Acad. Sci. U.S.A. 88:1339-1343.

13. Brown, P. O. Integration; Cold Spring Harbor Press: Cold Spring Harbor, 1999.

14. Asante-Appiah, E. and Skalka, A. M. (1999) Adv. Virus Res. 52:351-369.

15. Neamati, et al. (1997) DDT 2:487.

16. Neamati, N. (2001) Expert. Opin. Investig. Drugs 10:281.

17. Fesen et al. (1994) Biochem. Pharmacol. 48:595.

18. Marchand et al. (2001) Methods Enzymol. 340:624-633.

19. Dayam, R. and Neamati, N. (2003) Curr. Pharm. Des. 9:1789-1802.

20. Neamati, N. (2002) Expert Opin. Ther. Patent 12:709-724.

21. Stuerga, et al., (1993) Tetrahedron 49:6229-6234.

22. Stuyver et al., (2002) Antimicrob. Agents Chemother., 46:3854-3860.

23. Schinazi et al., (1990) Antimicrob. Agents Chemother. 34:1061-1067.

24. Patil et al. (2006) Bioorg. Med. Chem. 15:1212-1228.

25. Jiang et al. (2003) J. Org. Chem. 68:7555-7558.

26. Miyaura et al. (1981) Synth. Commun. 11:513.

27. Luliano et al. (2004) Org. Lett. 6, 3711-3714.

28. Furstner, A. and Mamane V. (2002) J. Org. Chem. 67:6264-6267.

29. Muller et al. (1996) Synleft, 521-522.

30. Corey, E. J. and Fuchs, P. L. (1972) Tetrahedron Lett. 3769-3772.

31. Mamane et al. (2004) Chem. Eur. J. 10:4556-4575.

32. Billingsley et al. (2006) Chem. Int. Ed. 45:3484-3488.

33. Mamane et al. (2004) J. Am. Chem. Soc. 126:8654-8655.

34. Radhakrishnan (1999) Tetrahedron Lett. 40:7533-7535.

35. Wang, D. Z. and Katz, T. J. (2005) J. Org. Chem. 70:8497-8502.

36. Habeeb et al. (2001) J. Med. Chem. 44,:2921-2927.

37. Zimmerman, H. E. and Wilson, W. W. (1995) J. Org. Chem. 60:692-697.

38. Wassmundt, F. W. and Kiesman, W. F. (1996) J. Org. Chem. 60:196-201

39. Nodiff et al. (1971) J. Med. Chem. 14:921-925.

40. Nordlander, J. E. and Njoroge, F. G. (1987) J. Org. Chem. 52:1627-1630.

Any patents or publications mentioned in this specification are indicative of the levels of those skilled in the art to which the invention pertains. These patents and publications are incorporated by reference herein to the same extent as if each individual publication was incorporated by reference specifically and individually. One skilled in the art will appreciate that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

What is claimed is:

1. A phenanthrene diketo acid (PKA) compound having the structure 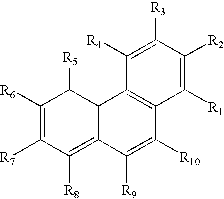
wherein R₁ is —C(O)CH₂C(O)COOH or H;
R₂ is H, F, —OCH₃, or NH₂;
R₃ is R₁ or F;
R₄ is R₂, R₃, OH, CH₃, or NHC(O)CH₃;
R₅ is H, F or C₁-C₄alkoxy;
R₆ is H, F, CN, C₁-C₄alkyl, C₁-C₄alkoxy, or O(CH₂)₃CN;
R₇ is H, F, CF₃, NO₂, C₁-C₄alkoxy, piperidyl, or methylpiperazine;
R₈ is H, NO₂ or C₁-C₄alkoxy;
R₉ is R₁; and
R₁₀ is CH₂OCH₂OCH₃, H, C₁-C₅alkyl, C₁-C₄alkoxy, tetrahydrofuran, 1-methyl-2,4-cyclopentene, pyrrolidine, or pyridine;
wherein one of R₁, R₂, R₃, R₄ or R₉ is C(O)CH₂C(O)COOH.

2. The PKA compound of claim 1, wherein the compound is a pharmacologically effective salt or hydrate thereof.

3. The PKA compound of claim 2, wherein the compound is a pharmaceutical composition further comprising a pharmaceutically effective carrier.

4. The PKA compound of claim 1, wherein R₁ is C(O)CH₂C(O)COOH, R₂ is H or NH₂, R₃ is H, R₄ is F, CH₃, OCH₃, R₅ is H or OCH₃, R₆ is H, CN, OCH₃, OCH₂CH₃, or O(CH₂)₃CN, R₇ is H, NO₂, CF3, CH₃, OCH₃, OCH₂CH₃, piperidyl, or methylpiperazine, R₈ is H or OCH₃, and R₉ and R₁₀ are H.

5. The PKA compound of claim 1, wherein R₁, R₅-R₆ and R₈-R₁₀ are H, R₂ is C(O)CH₂C(O)COOH, R₃ and R₄ are H or F, and R₇ is CH₃.

6. The PKA compound of claim 1, wherein R₁-R₂ and R₈-R₁₀ are H, R₃ is C(O)CH₂C(O)COOH, R4 is H or F, R5 is H, F, OCH₃, or O(CH2)3CH3, R₆ is H, F, CN, CH₃, CH(CH₃)₃, OCH₃, OCH₂(CH₃)₂, O(CH₂)₃CH₃, O(CH₂)₃CN, and R₇ is H, F, CF₃, NO₂, CH₃, OCH₃, piperidyl, or methylpiperazine.

7. The PKA compound of claim 1, wherein R₁-R₃, R₅-R₆ and R₈-R₁₀ are H, R₄ is C(O)CH₂C(O)COOH and R₇ is CH₃.

8. The phenanthrene diketo acid compound of claim 1, wherein R₁-R₈ are H, R₉ is C(O)CH₂C(O)COOH and R10 is CH₂OCH₂OCH₃, H, C₁-C₅alkyl, C₁-C₄alkoxy, tetrahydrofuran, 1-methyl-2,4-cyclopentene, pyrrolidine, or pyridine.

9. A method for inhibiting an activity of a human immunodeficiency viral (HIV) integrase protein, comprising:
contacting an HIV virus or a cell comprising HIV with a compound of claim 1 thereby inhibiting integrase protein activity therein.

10. The method of claim 9, wherein the HIV integrase activity is 3′-end processing or strand transfer of HIV RNA.

11. The method of claim 9, wherein the human immunodeficiency virus is HIV type 1 (HIV-1).

12. A method for treating an HIV infection in a subject, comprising:
administering to the individual a pharmacologically effective amount of one or more compounds of claim 1 to the subject thereby treating the HIV infection.

13. The method of claim 12, further comprising administering one or more other HIV antiviral drugs to the individual.

14. The method of claim 13, wherein the other HIV antiviral drug(s) is administered concurrently with or sequentially to the administration of the compound(s).

15. The method of claim 12, wherein the human immunodeficiency virus is HIV type 1 (HIV-1).

16. A method for identifying an inhibitor of HIV integrase protein, comprising:
designing a test compound based on the compounds of claim 1 as lead compounds;
measuring a level of an HIV integrase protein activity in the presence and the absence of the test compound; and
comparing the HIV integrase protein activity level in the presence of the test compound with the HIV integrase activity level in the absence of the test compound, wherein a decrease in HIV integrase protein activity in the presence of the test compound is indicative that the test compound is an inhibitor of HIV integrase protein.

17. The method of claim 16, further comprising determining a therapeutic index for the inhibitor.

18. The method of claim 16, wherein the HIV integrase activity is 3′-end processing or strand transfer of HIV RNA.

19. The method of claim 16, wherein the human immunodeficiency virus is HIV type 1 (HIV-1).

20. The inhibitory compound identified by the method of claim 16.

21. A synthetic inhibitor of HIV integrase protein comprising a phenanthrene ring substituted with a diketo acid moiety at one of C1-C4 and C9 on the phenanthrene, said phenanthrene ring further substituted with other than a hydrogen at one or more of carbons C1-C10.

22. The synthetic inhibitor of claim 21, wherein one or more of carbons C1-10 are substituted with one or more of fluorine, trifluoromethyl, nitrite, amine or alkylamine, alkyl or alkoxy or cyano-alkoxy, an alkyl diether, piperidyl, methylpiperazinyl, tetrahydrofuranyl, methylcyclopentenyl, pyrrolidinyl, or pyridinyl.


SEQUENCE LISTING (2 sequences)

SEQ ID NO: 1 - DNA, 21 bp, Artificial Sequence
  oligonucleotide used as a top sequence to assay HIV-1 integrase activity
gtgtggaaaatctctagcagt

SEQ ID NO: 2 - DNA, 21 bp, Artificial Sequence
  oligonucleotide used as a bottom sequence to assay HIV-1 integrase activity
actgctagagattttccacac